SEQUENCE LISTING (3 sequences)

SEQ ID NO: 1 - DNA, 24 bp, Artificial Sequence
  Synthetic construct
ttataactattcctnnnnnnnnnn

SEQ ID NO: 2 - DNA, 25 bp, Artificial Sequence
  Synthetic construct
nnnnnnnnnnctccctaataacaat

SEQ ID NO: 3 - DNA, 30 bp, Bacillus anthracis
taggaatagttataaattgttattagggag

(19) United States
(12) Patent Application Publication
(10) Pub. No.: US 2010/0120132 A1
(43) Pub. Date: May 13, 2010

(54) Bioassays by direct optical detection of nanoparticles

(75) Inventor: Tae-Woong Koo, Cupertino, CA (US)

Correspondence Address: Pillsbury Winthrop Shaw Pittman LLP;(INTEL), P.O. Box 10500, McLean, VA, 22102, US

(73) Assignee: Intel Corporation, Santa Clara, CA (US)

(21) Appl. No.: 11/394,157

(22) Filed: Mar. 31, 2006

(51) Int. Cl. G01N 33/543; G02B 21/10

(52) U.S. Cl. 435/287.2; 422/82.05; 435/288.7; 977/920; 977/835; 977/836; 359/385

(57) ABSTRACT

Embodiments of the invention relate to detecting biological molecules with ultra-sensitivity and convenience. The embodiments are especially directed to utilizing nanoparticles as tags and identifying the tags using dark-field microscopy. The probes containing the nanoparticles can be used in solution or attached to a substrate.

### FIELD OF INVENTION

The embodiments of the invention relate to methods and apparatus for detecting biological molecules with ultra-sensitivity and convenience. The embodiments are especially directed to utilizing nanoparticles as tags and identifying the tags using dark-field microscopy. The invention transcends several scientific disciplines such as polymer chemistry, biochemistry, molecular biology, medicine and medical diagnostics.

### BACKGROUND

The ability to detect and identify trace quantities of analytes has become increasingly important in virtually every scientific discipline, ranging from part per billion analyses of pollutants in sub-surface water to analysis of cancer treatment drugs in blood serum.

With the advancement of detection technologies, there are multiple techniques that promise biological detection with single molecule sensitivity. However, many of these techniques have not yet found commercial applications. The main reasons are the complexity associated with these ultra-sensitive methods. Many require multiple steps of chemical treatments, bulky and expensive instruments, and/or extreme care in sample handling and observation. These are not ideal for practical applications that require easy and reliable measurements.

In a dark field microscope, an opaque disk is placed underneath the condenser lens to prevent illumination light from directly going to the detector or viewer's eyes, therefore the background is completely dark. Only light that is scattered by objects in the sample can be detected. Dark field microscopy is suited for visualizing small scatters such as metal or semiconductor nanoparticles.

### BRIEF DESCRIPTION OF THE DRAWINGS

FIG. 1 is a schematic representation of using nanoparticles as a probe for protein detection.

FIG. 2 is a schematic representation of a nucleic acid assay.

FIG. 3 is a schematic representation of nucleic acid detection on a substrate surface.

FIG. 4 is a schematic representation of dark-field detection of nanoparticles.

FIG. 5 is a dark-field image of 20 nm gold nanoparticles (detected nanoparticles are marked with arrows).

FIG. 6 Schematic diagram of a hand-held diagnostic device.

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise.

An “array,” “macroarray” or “microarray” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray would generally contain spot sizes of less than 300 microns. A multiple-well array is a support that includes multiple chambers for containing sample spots.

“Solid support,” “support,” and “substrate” refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain aspects, the solid support(s) will take the form of beads, resins, gels, microspheres, or other geometric configurations.

The term “analyte”, “target” or “target molecule” refers to a molecule of interest that is to be analyzed. The analyte may be a Raman active compound or a Raman inactive compound. Further, the analyte could be an organic or inorganic molecule. Some examples of analytes may include a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The analyte molecule may be fluorescently labeled DNA or RNA.

The term “probe” or “probe molecule” refers to a molecule that binds to a target molecule for the analysis of the target. The probe or probe molecule is generally, but not necessarily, has a known molecular structure or sequence. The probe or probe molecule is generally, but not necessarily, attached to the substrate of the array. The probe or probe molecule is typically a nucleotide, an oligonucleotide, or a protein, including, for example, cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) The polynucleotide probes require only the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism. A probe or probe molecule can be a capture molecule.

The term “bi-functional linker group” refers to an organic chemical compound that has at least two chemical groups or moieties, such are, carboxyl group, amine group, thiol group, aldehyde group, epoxy group, that can be covalently modified specifically; the distance between these groups is equivalent to or greater than 5-carbon bonds.

The term “capture molecule” refers to a molecule that is immobilized on a surface. The capture molecule is generally, but not necessarily, binds to a target or target molecule. The capture molecule is typically a nucleotide, an oligonucleotide, or a protein, but could also be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to a target molecule that is bound to a probe molecule to form a complex of the capture molecule, target molecule and the probe molecule. The capture molecule may be fluorescently labeled DNA or RNA. The capture molecule may or may not be capable of binding to just the target molecule or just the probe molecule.

The terms “die,” “polymer array chip,” “DNA array,” “array chip,” “DNA array chip,” or “bio-chip” are used interchangeably and refer to a collection of a large number of probes arranged on a shared substrate which could be a portion of a silicon wafer, a nylon strip or a glass slide.

The term “chip” or “microchip” refers to a microelectronic device made of semiconductor material and having one or more integrated circuits or one or more devices. A “chip” or “microchip” is typically a section of a wafer and made by slicing the wafer. A “chip” or “microchip” may comprise many miniature transistors and other electronic components on a single thin rectangle of silicon, sapphire, germanium, silicon nitride, silicon germanium, or of any other semiconductor material. A microchip can contain dozens, hundreds, or millions of electronic components.

The term “molecule” generally refers to a macromolecule or polymer as described herein. However, arrays comprising single molecules, as opposed to macromolecules or polymers, are also within the scope of the embodiments of the invention.

“Predefined region” or “spot” or “pad” refers to a localized area on a solid support. The spot could be intended to be used for formation of a selected molecule and is otherwise referred to herein in the alternative as a “selected” region. The spot may have any convenient shape, e.g., circular, rectangular, elliptical, wedge-shaped, etc. For the sake of brevity herein, “predefined regions” are sometimes referred to simply as “regions” or “spots.” In some embodiments, a predefined region and, therefore, the area upon which each distinct molecule is synthesized is smaller than about 1 cm² or less than 1 mm², and still more preferably less than 0.5 mm². In most preferred embodiments the regions have an area less than about 10,000 μm² or, more preferably, less than 100 μm², and even more preferably less than 10 μm² or less than 1 μm². Additionally, multiple copies of the polymer will typically be synthesized within any preselected region. The number of copies can be in the hundreds to the millions. A spot could contain an electrode to generate an electrochemical reagent, a working electrode to synthesize a polymer and a confinement electrode to confine the generated electrochemical reagent. The electrode to generate the electrochemical reagent could be of any shape, including, for example, circular, flat disk shaped and hemisphere shaped.

“Micro-Electro-Mechanical Systems (MEMS)” is the integration of mechanical elements, sensors, actuators, and electronics on a common silicon substrate through microfabrication technology. While the electronics are fabricated using integrated circuit (IC) process sequences (e.g., CMOS, Bipolar, or BICMOS processes), the micromechanical components could be fabricated using compatible “micromachining” processes that selectively etch away parts of the silicon wafer or add new structural layers to form the mechanical and electromechanical devices. Microelectronic integrated circuits can be thought of as the “brains” of a system and MEMS augments this decision-making capability with “eyes” and “arms”, to allow microsystems to sense and control the environment. Sensors gather information from the environment through measuring mechanical, thermal, biological, chemical, optical, and magnetic phenomena. The electronics then process the information derived from the sensors and through some decision making capability direct the actuators to respond by moving, positioning, regulating, pumping, and filtering, thereby controlling the environment for some desired outcome or purpose. Because MEMS devices are manufactured using batch fabrication techniques similar to those used for integrated circuits, unprecedented levels of functionality, reliability, and sophistication can be placed on a small silicon chip at a relatively low cost.

“Microprocessor” is a processor on an integrated circuit (IC) chip. The processor may be one or more processor on one or more IC chip. The chip is typically a silicon chip with thousands of electronic components that serves as a central processing unit (CPU) of a computer or a computing device.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or ω-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure.

The terms “nanomaterial” and “nanoparticles” as used herein refers to a structure, a device or a system having a dimension at the atomic, molecular or macromolecular levels, in the length scale of approximately 1-100 nanometer range. Preferably, a nanomaterial has properties and functions because of the size and can be manipulated and controlled on the atomic level. Nanoparticles made of semiconducting material may also be labeled quantum dots if they are small enough (typically sub 10 nm) that quantization of electronic energy levels occurs. Preferred nanoparticles as used herein are metallic nanoparticles. More preferred nanoparticles that include coinage (Au, Ag, Cu), alkalis (Li, Na, K), Al, Pd and Pt.

A “carbon nanotube” refers to a fullerene molecule having a cylindrical or toroidal shape. A “fullerene” refers to a form of carbon having a large molecule consisting of an empty cage of sixty or more carbon atoms.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogens of the purine and pyrimidine moiety are protected by groups such as dimethoxytrityl, benzyl, tert-butyl, isobutyl and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary polynucleotides (especially RNA) than their unmodified counterparts. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the polynucleotide can also be substituted or modified, for instance with methyl phosphonates, O-methyl phosphates or phosphororthioates. Another example of a polynucleotide comprising such modified linkages for purposes of this disclosure includes “peptide polynucleotides” in which a polyamide backbone is attached to polynucleotide bases, or modified polynucleotide bases. Peptide polynucleotides which comprise a polyamide backbone and the bases found in naturally occurring nucleotides are commercially available.

Nucleotides with modified bases can also be used in the embodiments of the invention. Some examples of base modifications include 2-aminoadenine, 5-methylcytosine, 5-(propyn-1-yl)cytosine, 5-(propyn-1-yl)uracil, 5-bromouracil, 5-bromocytosine, hydroxymethylcytosine, methyluracil, hydroxymethyluracil, and dihydroxypentyluracil which can be incorporated into polynucleotides in order to modify binding affinity for complementary polynucleotides.

Groups can also be linked to various positions on the nucleoside sugar ring or on the purine or pyrimidine rings which may stabilize the duplex by electrostatic interactions with the negatively charged phosphate backbone, or through interactions in the major and minor groves. For example, adenosine and guanosine nucleotides can be substituted at the N² position with an imidazolyl propyl group, increasing duplex stability. Universal base analogues such as 3-nitropyrrole and 5-nitroindole can also be included. A variety of modified polynucleotides suitable for use in the embodiments of the invention are described in the literature.

When the macromolecule of interest is a peptide, the amino acids can be any amino acids, including α, β, or ω-amino acids. When the amino acids are a-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it will be recognized by those of skill that the minimum length of a polynucleotide desired for specific hybridization to a target polynucleotide will depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions will vary depending on the application and are selected in accordance with the general binding methods known in the art.

It is appreciated that the ability of two single stranded polynucleotides to hybridize will depend upon factors such as their degree of complementarity as well as the stringency of the hybridization reaction conditions.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, will remain single-stranded. Conversely, at lower stringency, the probability of formation of a mismatched duplex is increased.

The appropriate stringency that will allow selection of a perfectly-matched duplex, compared to a duplex containing one or more mismatches (or that will allow selection of a particular mismatched duplex compared to a duplex with a higher degree of mismatch) is generally determined empirically. Means for adjusting the stringency of a hybridization reaction are well-known to those of skill in the art.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors maybe naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “Ligand Receptor Pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies (Abs): For instance, the invention may be useful in investigating the ligand-binding site on the antibody molecule which combines with the epitope of an antigen of interest; determining a sequence that mimics an antigenic epitope may lead to the-development of vaccines of which the immunogen is based on one or more of such sequences or lead to the development of related diagnostic agents or compounds useful in therapeutic treatments such as for auto-immune diseases (e.g., by blocking the binding of the “anti-self” antibodies). There are monoclonal antibodies (mAb) and polyclonal antibodies (pAb).

d) Nucleic Acids: Sequences of nucleic acids may be synthesized to establish DNA or RNA binding sequences. Certain sequence of nucleic acids, called aptamer, can bind to proteins or peptides.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

A “linker” molecule refers to any of those molecules described supra and preferably should be about 4 to about 100 atoms long to provide sufficient exposure. The linker molecules may be, for example, aryl acetylene, alkane derivatives, ethylene glycol oligomers containing 2-10 monomer units, diamines, diacids, amino acids, among others, and combinations thereof. Alternatively, the linkers may be the same molecule type as that being synthesized (i.e., nascent polymers), such as polynucleotides, oligopeptides, or oligosaccharides.

The phrase “SERS active particle refers” to particles that produce the surface-enhanced Raman scattering effect. The SERS active particles generate surface enhanced Raman signal specific to the analyte molecules when the analyte-SERS complexes are excited with a light source. The enhanced Raman scattering effect provides a greatly enhanced Raman signal from Raman-active analyte molecules that have been adsorbed onto certain specially-prepared SERS active particle surfaces. Typically, the SERS active particle surfaces are metal surfaces. Increases in the intensity of Raman signal have been regularly observed on the order of 10⁴-10¹⁴ for some systems. SERS active particles include a variety of metals including coinage (Au, Ag, Cu), alkalis (Li, Na, K), Al, Pd and Pt.

The term “fluid” used herein means an aggregate of matter that has the tendency to assume the shape of its container, for example a liquid or gas. Analytes in fluid form can include fluid suspensions and solutions of solid particle analytes.

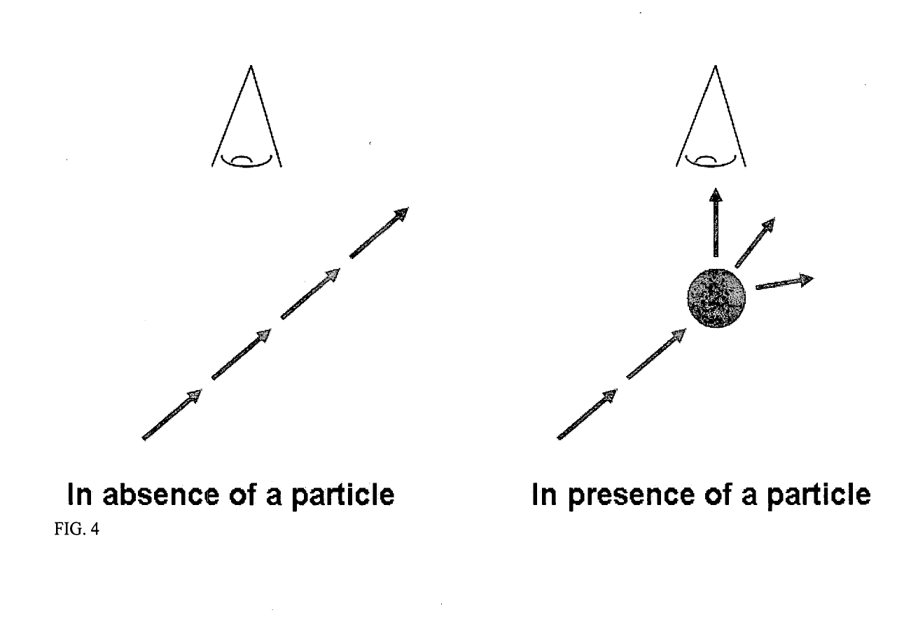

Dark-field microscopy relies on a different illumination system than standard brightfield microscopy. Rather than illuminating the sample with a filled cone of light, the condenser in a dark-field microscope is designed to form a hollow cone of light. The light at the apex of the cone is focused at the plane of the specimen; as this light moves past the specimen plane it spreads again into a hollow cone. The objective lens sits in the dark hollow of this cone; although the light travels around and past the objective lens, no rays enter it. The entire field appears dark when there is no sample on the microscope stage; thus the name dark-field microscopy. When a sample is on the stage, the light at the apex of the cone strikes it. As shown in FIG. 4, the image is made only by those rays scattered by the sample and captured in the objective lens (note the rays scattered by the particle in FIG. 4). The image appears bright against the dark background.

Dark-field microscopes are typically equipped with specialized condensers constructed only for dark-field application. This dark-field effect can be achieved in a brightfield microscope, however, by the addition of a simple “stop”. The stop is a piece of opaque material placed below the substage condenser; it blocks out the center of the beam of light coming from the base of the microscope and forms the hollow cone of light needed for dark-field illumination.

Dark-field microscopy reduces the amount of light entering the lens system of a microscope in two ways. First, the stop blocks the center of the beam of light that would otherwise fill the objective lens. Second, only the light which is scattered by the specimen and enters the objective lens is seen. Therefore, the best viewing result typically requires increasing the light intensity as much as possible: by setting the light intensity adjustment at maximum, by opening the field diaphragm, by opening the condenser aperture, and by removing any color or other filters. The particle container preferably holds the particle sample within the field of view of the microscope.

The use of dark-field microscopy provides rapid and direct visualization of individual particles. Accordingly, this procedure can be used to achieve high efficiency and accuracy on samples in the colloidal state. When using TEM, samples typically need to be dried on a thin film, which often results in aggregation of nanoparticles, making it difficult to determine the original particle concentration. In addition, the time and complexity for obtaining one dark-field image is orders of magnitude less than those for obtaining one TEM image.

Magnetic probes are particles whose core is made of magnetic materials, such as compounds containing iron, palladium, platinum, aluminium, barium, calcium, sodium, strontium, uranium, magnesium, cobalt, nickel, or technetium. Magnetic probes show attraction to magnets, but are not magnets by themselves. The surface of the particle can be first chemically treated (e.g. silanization) to facilitate the binding of the probe molecule. The surface of the particle is coated with the probe molecules, such as DNA or antibodies.

Embodiments of the invention relate to detecting biological molecules with ultra-sensitivity and convenience. The embodiments are especially directed to utilizing nanoparticles as tags and identifying the tags using dark-field microscopy. The probes containing the nanoparticles can be used in solution or attached to a substrate.

One embodiment is a method of detecting an analyte with a nanoparticle probe. The method includes attaching a detection probe including a nanoparticle to a target analyte, and detecting the presence of the nanoparticle utilizing dark-field microscopy.

The target analyte is preferably a biomolecule. More preferably, the target analyte is a nucleic acid, protein, or an antibody. Preferably, the detection probe comprises nucleic acid. Preferably, the nanoparticle includes a metal. Preferred metals include Au, Ag, Cu, Al, Pd and Pt. The detection probe may also include a linker molecule.

Preferably, the method also includes separating the attached detection probe and target analyte from unattached detection probes prior to detecting the presence of the nanoparticle utilizing dark-field microscopy. Preferably, the method includes attaching a magnetic probe to the target analyte. Preferably, the magnetic probe includes a magnetic particle and a capture molecule. Preferably, the method also includes exposing the target analyte to a magnetic field to separate the target analyte attached to the magnetic probe from unattached detection probes prior to detecting the presence of the nanoparticle utilizing dark-field microscopy. Preferably, the nanoparticle attached to the target analyte is separated from the magnetic probe prior to detecting the presence of the nanoparticle utilizing dark-field microscopy.

Another embodiment of a method of detecting an analyte with a nanoparticle probe includes attaching a target analyte to a substrate, attaching a detection probe including a nanoparticle to the target analyte, and detecting the presence of the nanoparticle utilizing darkfield microscopy.

Preferably, the nanoparticle may be attached to the substrate prior to detecting the presence of the nanoparticle. Preferably, the nanoparticle is released from the substrate prior to detecting the presence of the nanoparticle. Preferably, the substrate is includes glass, silicon, gold, platinum, or polymers.

The target analyte is preferably a biomolecule. More preferably, the target analyte is a nucleic acid, protein, or an antibody. Preferably, the detection probe comprises nucleic acid. Preferably, the nanoparticle includes a metal. Preferred metals include Au, Ag, Cu, Al, Pd and Pt. The detection probe may also include a linker molecule.

Preferably, the method also includes separating the attached detection probe and target analyte from unattached detection probes prior to detecting the presence of the nanoparticle utilizing dark-field microscopy. Preferably, a capture molecule attaches the target analyte to the substrate. The capture molecule preferably includes a biomolecule. More preferably, the capture molecule includes an antibody, nucleic acid or an aptamer. Preferably the capture molecule is attached to the substrate via a linker.

Another embodiment is a system including a detection probe capable of binding to a target analyte, wherein the detection probe includes a nanoparticle. The system also includes a dark-field microscope configured to detect the nanoparticle.

Preferably, the system also includes a magnetic probe capable of binding to a target analyte and a magnet configured to separate the target analyte attached to the magnetic probe from unattached detection probes prior to detecting the presence of the nanoparticle utilizing dark-field microscopy. Preferably, the magnetic probe comprises a magnetic particle and a capture molecule.

Yet another embodiment is a device that includes a substrate, and a capture molecule attached to the substrate, wherein the capture molecule is capable of binding to a target analyte. The device also includes a detection probe capable of binding to the target analyte, wherein the detection probe comprising a nanoparticle, and a dark-field microscope.

Preferably, the nanoparticle is attached to the substrate prior to detecting the presence of the nanoparticle. Preferably, the nanoparticle is released from the substrate prior to detecting the presence of the nanoparticle.

Nanoparticles, particularly metallic nanoparticles, are very good at scattering light and are therefore easy to identify using dark-field microscopy. Since it is possible to detect small nanoparticles using a dark field microscopy, it has been found that nanoparticles can be used as a tag for a probe. For example, nanoparticles can be used as a biological assay. Described are reliable and simple methods for utilizing nanoparticles as tags. The methods can be used with or without any additional steps to enlarge the nanoparticles before detection. This allows for simpler and faster detection of probe molecules.

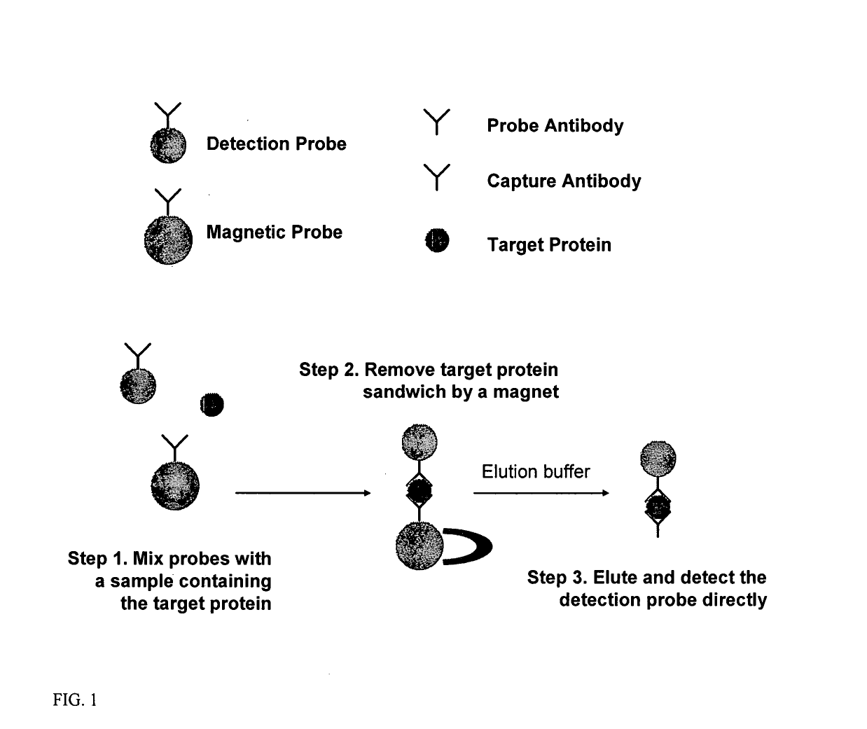

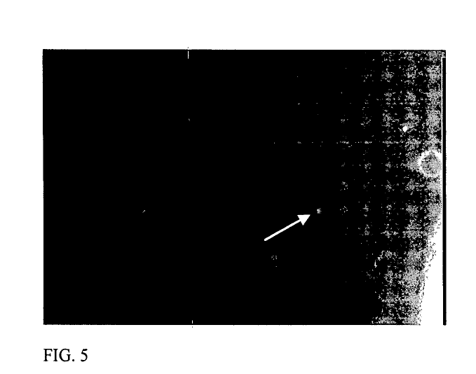

FIG. 1 shows an embodiment for using nanoparticles as a probe for protein detection. In this embodiment, 20 nm gold nanoparticles are used as detection probes. The detection probes are coated with probe antibodies. Magnetic probes are coated with capture antibodies. Probe antibodies and capture antibodies bind to the target protein at a specific location. When the detection probes and the magnetic probes are mixed with a sample containing the target protein in Step 1, they form a complex, which called a sandwich (the target protein is bound between the capture antibody and the probe antibody). This sandwich can be separated from excess material and contaminants using a magnet, since magnetic probes are attracted to the magnet in Step 2. After washing the sandwich to remove excess material, an elution buffer is added to release the detection probes from magnetic probes in Step 3. The released detection probes can then be observed by dark-field observation as shown in FIG. 5, which is a dark-field image of 20 nm gold nanoparticles (detected nanoparticles are marked with arrows).

### Example 1

### Protein Detection

Following is an example of detecting a small soluble protein in cerebral spinal fluid (CSF) known as amyloid-beta-derived diffusible ligand (ADDL). The presence of ADDL in CSF samples has been associated with Alzheimer's disease. Although this example is specific for ADDL detection, the same method can be used for the detection of a variety of proteins and other biological assays.

CSF sample Preparation—CSF samples can be obtained via lumbar puncture and kept frozen until used.

Antigen Isolation and Antibody (Ab) Expression for ADDLs—Aβ₁₋₄₂ peptide (California Peptide Research, Napa, Calif.) is used to prepare synthetic ADDLs according to known protocols. An aliquot of A(_{—}1-42 is dissolved in anhydrous DMSO to a concentration of 22.5 mg/ml (5 mM), pipette-mixed, and further diluted into ice-cold F12 medium (phenol-red-free) (1:50 dilution; BioSource International, Camarillo, Calif.). The mixture can be quickly vortexed, incubated at 6-8° C. for 24 hours, and centrifuged at 14,000×g for 10 minutes, and the oligomers can be collected from the supernatant. The concentration of synthetic ADDLs can be determined by using a microBCA assay (Pierce, Rockford, Ill.). Abs targeting ADDLs in the bio-barcode assay (M90 pAb and 20C2 mAb) can then be generated and characterized by methods known in the art (e.g. See Lambert, M. P., Viola, K. L., Chromy, B. A., Chang, L., Morgan, T. E., Yu, J., Venton, D. L., Krafft, G. A., Finch, C. E., Klein, W. L. (2001) J. Neurochem. 3, 595-605).

Nucleoprotein Nanoparticle (NP) Synthesis and Modification—Citrate-stabilized detection probes can be prepared by following standard methods known in the art (e.g. See Jin, R. C., Wu, G. S., Li, Z., Mirkin, C. A. & Schatz, G. C. (2003) J. Am. Chem. Soc. 125, 1643-1654.). and ferromagnetic probes (0.5 nM) Nanoparticles can be purchased from commercial vendors, such as BB International (Cardiff, U.K.). The nanoparticles (1 ml) can be initially functionalized with 1 μg of antigen-specific Ab (M90) in a basic aqueous solution (pH 9). The particles can be centrifuged at 15,700×g, and the supernatant containing excess antibody can be removed. The particles can then be resuspended in 0.1 M PBS, and the procedure can be repeated three times to ensure the removal of excess antibodies, and we call the nanoparticle-antibody complex “detection probe.” The concentration of the nanoparticles can be calculated based on extinction spectra by using known values of the extinction coefficients for the nanoparticles. The diameters of the synthesized nanoparticles can be determined by transmission electron microscopy by using a commercially available instrument (e.g. Model 8100, manufacture by Hitachi, Tokyo).

Functionalization of Magnetic Probe—The amino-functionalized magnetic particles (MPs) (100 μl, 50 mg/ml aqueous solution, 1 μm diameter polyamine particles with iron oxide cores manufacturered by Polysciences) can be modified with 100 μg of antibodies according to the manufacturer's protocol. The Abs can be mAbs specific to ADDL (20C2). We call the magnetic particle—antibody complex “magnetic probe.”

Bio-Barcode Assay—In a typical assay, 10 μl of CSF or 10-μl aliquots of ADDL at known concentrations ranging from 100 aM to 100 fM can be added to 50 μl of magnetic probe solution (5 mg/ml) and allowed to react under vigorous stirring at 37° C. for 1 h. After magnetically immobilizing the magnetic probes, the unbound antigens can be removed by repeated washing with PBS. The magnetic probes and antigen-target complexes can be magnetically separated, and 50 μl of 0.1 nM detection probe (Ab-functionalized) can be added and stirred vigorously at 37° C. for 30 min to bind the target-antigen-magnetic probe complex. The sandwich complexes can be then magnetically separated and washed four times with 100 μl of PBS solution. In the final step, 50 μl of elution buffer (0.1 M glycine-HCl, pH 2.5) can be added and the solutions can be stirred vigorously for 30 min to allow for full elution of the antibody and/or antigen. The remaining complexes can again be separated magnetically, and the supernatant containing the gold nanoparticles can be collected for quantification.

Darkfield Detection—Light scattering by the gold particles can be quantified directly in solution with a dark field microscope (manufactured, for example, by NIKON USA) and the scattering image of the whole slide can be collected. The concentration of the particles can be determined by counting the total number of particles in one image and dividing this number by the volume (thickness×width×height) of the visible sample.

Unlike previous detection techniques DNA synthesis and DNA immobilization to the nanoparticles are not necessary, because this method does not require DNA barcoding. In addition no amplification of the signal is necessary. Further, a nanoparticles enlargement step is not necessary, because the nanoparticles can be detected without enlargement.

### Example 2

### Nucleic Acid Detection

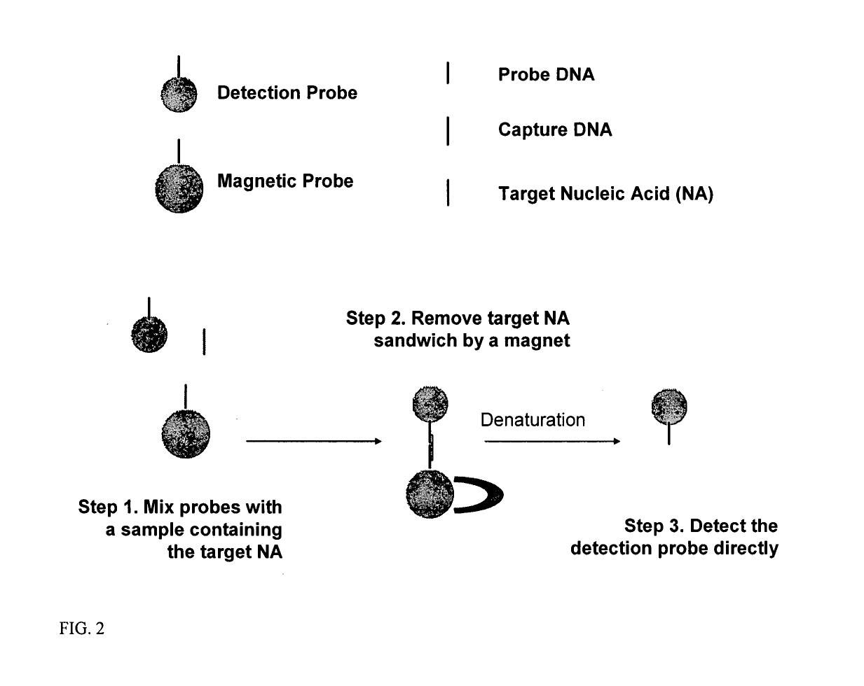

Following is an example of detecting a small soluble nucleic acid in accordance with the method described with reference to FIG. 2.

DNA Synthesis—DNA strands can be synthesized and purified according to standard procedure using an automated synthesizer (Expedite) and HPLC (1100 HPLC series, Hewlett-Packard), respectively. The reagents for the phosphoramidite synthesis, including 3_- and 5_-thiol modifiers, can be purchased from Glen Research (Sterling, Va.). Thiol modification can be carried out manually by following standard procedures. Absorption and extinction spectra can be recorded by using an 8452a diode array spectrophotometer (Hewlett-Packard). The concentrations of stock DNA solutions are calculated based on the extinction coefficient of each strand. All buffers and aqueous washes are based on Nanopure water (18 MΩ; Barnstead), and reagents can be used as received unless indicated otherwise. The following DNA strands are synthesized for the nucleic acid assay: probe DNA, 5_-TTATAACTATTCCTA10-(CH2)6-SH-3; capture DNA, 5_-HS-(CH2)6-A10-CTCCCTAATAACAAT-3; both are designed to bind to part of the target, 5_-TAGGAATAGTTATAAATTGTTATTAGGGAG-3.

Functionalization of Detection Probe—Au nanoparticles can be purchased and used as received from BB International (Cardiff, U.K.). The Au particles are modified with thiolated probe DNA (final concentration, 2 (M) by slow salt aging (40 h) to a final concentration of PBS (0.1 M NaCl in 0.01 M of phosphate buffer, pH 7; denoted as PBS unless indicated otherwise). Unbound probe DNA is removed by repetitive centrifugation (15,700×g for 30 min) of the particles, followed by rinsing and resuspension in PBS. This consist the detection probes. The concentration of the detection probes is calculated based on extinction spectra by using known values of the extinction coefficients. The diameter of the synthesized detection probe is determined by transmission electron microscopy by using a commercially available instrument (e.g. Model 8100 manufactured by Hitachi, Tokyo).

Functionalization of Magnetic Probe. The gold coated paramagnetic particles can be modified with thiolated capture DNA (final concentration, 2 (M) by slow salt aging (40 h) to a final concentration of PBS). Unbound capture DNA is removed by repetitive centrifugation (15,700×g for 30 min) of the particles, followed by rinsing and resuspension in PBS. Alternatively, amine functionalized magnetic particles can be reacted with thiolated capture DNA. We call the magnetic particle-capture DNA complex “magnetic probe.” The concentration of the particles is calculated based on extinction spectra by using known values of the extinction coefficients. The diameter of the synthesized magnetic probe is determined by transmission electron microscopy by using an 8100 instrument (Hitachi, Tokyo).

Bio-Barcode Assay—In a typical assay, 10 μl of sample suspected of containing the target DNA is added to 50 μl mixture of magnetic probe solution (5 mg/ml) and detection probe solution (5 mg/ml) and allowed to incubate at room temperature for 4 h. After magnetically immobilizing the magnetic probe, the unbound target DNA and the unbound detection probe is removed by repeated washing with PBS. In the final step, 50 p. 1 of H₂O is added and the solutions is stirred vigorously at 60° C. for 30 min to allow for full dehybridization of the target DNA and the detection probe. The complex is again separated magnetically, and the supernatant containing the detection probe can be collected for quantification by the darkfield detection and quantification.

Darkfield Detection—Light scattering by the gold particles is quantified directly in solution with a dark field microscopy (Nikon USA) and the scattering image of the whole slide can be collected. The concentration of the particles can be determined by counting the total number of particles in one image and dividing this number by the volume (thickness×width×height) of the visible sample.

### Example 3

### Nucleic Acid Detection on a Substrate Surface

When multiple analytes are to be detected in the same sample, capture antibodies or capture DNA can be immobilized on a substrate. By spotting different capture antibodies or DNA onto different physical locations and by monitoring where the detection probes are bound, we can measure which biological molecules are present in the sample.

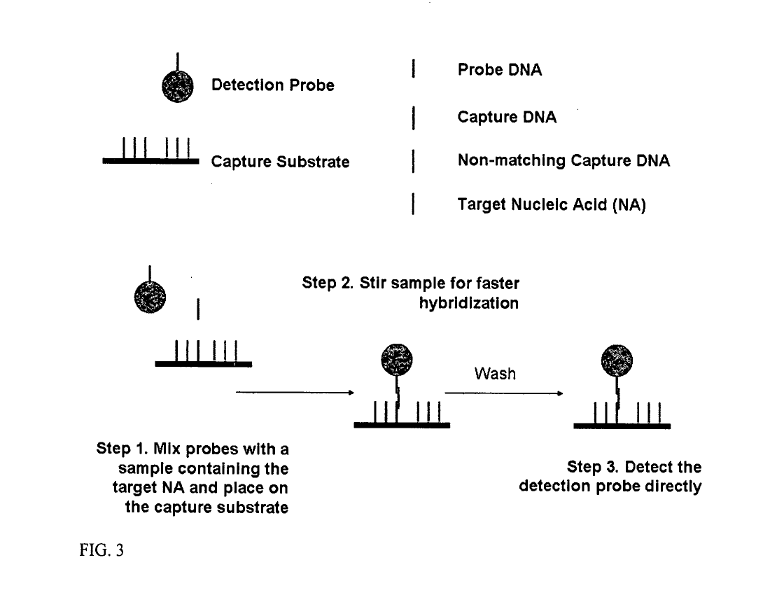

Following is an example of detecting a small soluble nucleic acids using capture DNA secured to a substrate surface in accordance with the method described with reference to FIG. 3.

DNA Synthesis—DNA strands are synthesized and purified according to standard procedures using an automated synthesizer (Expedite) and HPLC (1100 HPLC series, Hewlett-Packard), respectively. All of the reagents for the phosphoramidite synthesis, including 3_- and 5_-thiol modifiers, can be purchased from Glen Research (Sterling, Va.). Thiol modification is carried out manually by following known procedures. Absorption and extinction spectra are recorded by using an 8452a diode array spectrophotometer (Hewlett-Packard). The concentrations of stock DNA solutions are calculated based on the extinction coefficient of each strand. All buffers and aqueous washes are based on Nanopure water (18 M(; Barnstead), and reagents are used as received unless indicated otherwise. The following DNA strands are synthesized for the nucleic acid assay: probe DNA, 5_-TTATAACTATTCCTA10-(CH2)6-SH-3; capture DNA, 5_-HS-(CH2)6-A10-CTCCCTAATAACAAT-3; both are designed to bind to part of the target, 5_-TAGGAATAGTTATAAATTGTTATTAGGGAG-3.

Functionalization of Detection Probe—Au nanoparticles can be purchased and used as received from BB International (Cardiff, U.K.). The Au particles can be modified with thiolated probe DNA (final concentration, 2 (M) by slow salt aging (40 h) to a final concentration of PBS (0.1 M NaCl in 0.01 M of phosphate buffer, pH 7; denoted as PBS unless indicated otherwise). Unbound probe DNA can be removed by repetitive centrifugation (15,700×g for 30 min) of the particles, followed by rinsing and resuspension in PBS. The concentration of the detection probes can be calculated based on extinction spectra by using known values (28) of the extinction coefficients. The diameter of the detection probes can be determined by transmission electron microscopy by using a commercially available instrument (e.g. Model 8100 manufactured by HITACHI, Tokyo).

Functionalization of Glass Slides—Functionalized glass slides are modified with half-complementary thiolated capture DNA strands (100 (M) using a microarrayer (AFFYMETRIX, Santa Clara, Calif.) according to a standard procedures. The DNA strands are covalently immobilized on the chip, the unbound strands are washed away with H₂O, and the residual binding sites are passivated by immersion in 40 mM mercaptosuccinic acid for 30 min, followed by repetitive washing with H₂O.

Bio-Barcode Assay—In a typical assay, the glass slide is treated with the sample suspected of containing the target DNA to allow the target DNA to partially hybridize to the capture DNA. After 4 hour incubation at room temperature, the slide is washed with PBS four times to remove the sample and unbound DNA. The glass slide is then treated with the probe solution (5 mg/ml) to allow the detection probe to partially hybridize to the target DNA already partially hybridized to the capture DNA. After 4 hour incubation at room temperature, the slide is again washed with PBS four times to remove unbound detection probes. The slide can be dried with dry nitrogen gas for darkfield detection. Alternatively, the slide can be directly scanned by darkfield field microscopy without drying.

Darkfield Detection—Light scattering by the gold particles can be quantified directly in solution with a dark field microscopy (Nikon USA) and the scattering image of the whole slide can be collected.

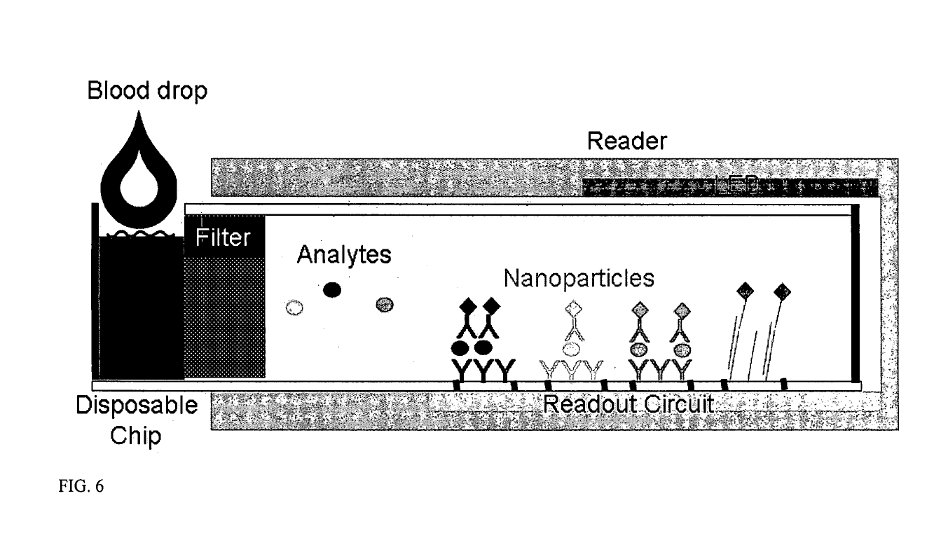

FIG. 6 shows an example of a handheld diagnostic device that utilizes capture molecules bound to a substrate surface. It combines a disposable chip containing biochemical reagents and microfluidic devices. When a sample (e.g. blood) is put onto the chip, the cells in the sample are lysed and DNA is released. Also proteins present in the blood can pass through the filter. When these molecules reach the reaction chamber, where the detection probes are present, sandwiches form. The handheld device has a light source, such as a light emitting diode (LED), which illuminates the sample at an oblique angle. An array detector integrated with the read-out circuit on the bottom side of the device has a limited acceptance angle, and light from the LED cannot be read by the detector unless a nanoparticle in the sandwich scatters light. The intensity of the detected light, the location of detection, and the timing of detection are processed by a microprocessor to tell the presence of the target molecule in the sample to the user.

The biochip preferably includes a plastic case, top window, bottom substrate, a filter, and reagents. The plastic case has multiple channels inside to allow liquid to flow and houses the filter and reagents as well as the bottom substrate. The plastic case also has a well to allow a liquid sample to be deposited. The filter contains chemicals to lyse the cells and release nucleic acids in the sample, and allows the molecules of interest to pass through. Such filters can be found in i-Stat chips (manufactured by Johnson & Johnson). Reagents include the detection probe as well as buffers. The buffers can be moved inside the chip microfluidically. The substrate is made of optically transparent material (e.g. glass, polymer, or silicon derivatives) and is pre-spotted with antibodies or capture DNAs of interest at desired locations. The top window is made of optically transparent material (e.g. glass, plastic, polymer, or silicon derivatives).

The reader preferably includes a light source, a detector, electronics, and microfluidic actuators. The light source can be a light-emitting-diode (LED), a lamp (mercury, halogen, or xenon), a fluorescent light source, an incandescent light source, or a chemiluminescent or electroluminescent source. The detector can be a charge-coupled-device (CCD), a complementary metal-oxide semiconductor (CMOS) detector, a photodiode, an avalanche photodiode, or plurality of photodiodes or avalanche photodiodes. The microfluidic actuators apply force or pressure to move liquids inside the chip. The electronics controls the time and sequence of the microfluidic actuation as well as activating the light source, reading out the optical signal from the detector, processing the signal numerically, and storing, transferring, and displaying the result. The reader may have a display where the device operation status and result can be displayed. The reader may also have an electrical connection to allow interfacing with other electrical/electronic devices, including a personal computer. The reader may also have an interface to allow data storage in an external data storage device such as a universal serial bus (USB) memory drive or an external harddrive. The reader may have a network connection to transfer the data over a local area network (LAN) or via Ethernet protocol.

In operation of the device, a sample, such as a drop of blood, is dropped into the well in the chip. Then the chip is plugged into the reader, and the reader operation is initiated. The electronics in the reader activates microfluidic actuators for sample processing, and optical components for signal detection. The result may be displayed on the display unit or downloaded separately.

The devices and methods described herein can be used for a variety of applicants, for example, in the point of care and field devices for diagnostics, forensic, pharmaceutical, agricultural, food inspection, biodefense, environmental monitoring, and industrial process monitoring.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1.-64. (canceled)

65. A device comprising a non-magnetic detection probe comprising a nanoparticle and a probe molecule attached to the nanoparticle, wherein the detection probe is in suspension in a liquid buffer and capable of binding to a target analyte; a magnetic capture probe comprising a magnetic particle and a capture molecule, wherein the device is configured to mix the non-magnetic detection probe and the magnetic capture probe with the target analyte to form a complex comprising the non-magnetic detection probe, the target analyte and the magnetic capture probe; wherein the device is configured to separate the complex with a-magnet; and wherein the device is configured to at least release the magnetic particle from the complex to form a released non-magnetic detection probe, wherein the device is configure to detect the presence of the nanoparticle with dark field microscopy.

66. The device of claim 65, further comprising a filter containing chemicals of lyse cells and release the target analyte, and the filter has a structure to allow the target analyte to pass through the filter.

67. The device of claim 65, further comprising an elution buffer to elute the magnetic particle of the magnetic probe.

68. The device of claim 65, further comprising a light source, and a detector configured to detect the nanoparticle.

69. The device of claim 65, wherein the target analyte is a biomolecule.

70. The device of claim 65, wherein the target analyte is a nucleic acid.

71. The device of claim 65, wherein the target analyte is a protein.

72. The device of claim 65, wherein the probe molecule or the capture molecule comprises an antibody.

73. The device of claim 65, wherein the probe molecule or the capture molecule comprises nucleic acid.

74. The device of claim 65, wherein the nanoparticle comprises a metal.

75. The device of claim 65, wherein the nanoparticle comprises a metal selected from the group consisting of Ag, Ag, Cu, alkalis, Al, Pd or Pt.

76. The device of claim 65, wherein the detection probe comprises a linker molecule.

77. The device of claim 65, wherein the detection probe is freely in suspension in a liquid buffer and capable of moving in the liquid buffer and binding to the target analyte.

78. A device comprising a non-magnetic detection probe comprising a nanoparticle and a probe molecule attached to the nanoparticle, wherein the detection probe is in suspension in a liquid buffer and capable of binding to a target analyte; a magnetic capture probe comprising a magnetic particle and a capture molecule, wherein the device is configured to mix the non-magnetic detection probe and the magnetic capture probe with the target analyte to form a complex comprising the non-magnetic detection probe, the target analyte and the magnetic capture probe; wherein the device is configured to separate the complex with a magnet; and wherein the device is configured to at least release the magnetic particle from the complex to form a released non-magnetic detection probe; and a dark field microscope.
